(19) United States
(12) Patent Application Publication
(10) Pub. No.: US 2017/0014073 A1
(43) Pub. Date: Jan. 19, 2017

(54) ELASTIC CONDUCTIVE STRIPE AND METHODS OF UTILIZING THEREOF

(71) Applicant: HEALTHWATCH LTD., Herzliya (IL)

(72) Inventors: Boaz SHOSHANI, Raanana (IL); Uri AMIR, Or Yehuda (IL)

(73) Assignee: HEALTHWATCH LTD., Herzliya (IL)

(21) Appl. No.: 15/121,334

(22) PCT Filed: Mar. 5, 2015
(86) PCT No.: PCT/IL2015/050239

§ 371 Date: Aug. 24, 2016

Related U.S. Application Data
(60) Provisional application No. 61/950,139, filed on Mar. 9, 2014.
(60) Provisional application No. 62/006,102, filed on May 31, 2014.

(51) Int. Cl. A61B 5/00; A61B 5/0428; A41D 27/10; A41D 1/00; A41D 1/04

(52) U.S. Cl. CPC A61B 5/6805; A41D 1/005; A41D 1/04; A41D 27/10; A61B 5/04286; A41D 2500/10; A61B 2560/0456; A61B 2560/0468; A61B 2562/0209; A41D 2300/322

(57) ABSTRACT

According to the teachings of the present invention there is provided a knitted smart garment. The garment includes a tubular form having variable elasticity and at least one conductive textile electrode for sensing an electrical vital signal, such as a clinical-level ECG signal. The garment further includes at least one elastic and loose conductive stripe, having a first end and a second end. The first end of the at least one conductive stripe is securely attached to a respective conductive textile electrode, and the second end of the at least one conductive stripe is operatively connected with a processor. The elasticity and looseness of the at least one conductive stripe is configured to prevent a pulling force from being applied to the respective conductive textile electrode, when the garment is stretched.

### CROSS REFERENCE TO RELATED APPLICATIONS

This application claims the benefit under 35 USC 119(e) from U.S. provisional application 61/950,139 filed Mar. 9, 2014, and the benefit under 35 USC 119(e) from U.S. provisional application 62/006,102 filed May 31, 2014, the disclosure of which are included herein by reference.

This application also relates to the PCT/IL2013/050963 ('963), the disclosure of which is included herein by reference in its entirety.

### FIELD OF THE INVENTION

The present invention relates to real-time health monitoring systems and more particularly, the present invention relates to a knitted garment having a tubular form at preconfigured locations, transferring ECG or other signals from textile electrodes to a selected area of the garment.

### BACKGROUND OF THE INVENTION AND PRIOR ART

Monitoring systems for monitoring of physiological parameters of a living being are well known in prior art. For example, PCT/IL2012/000248, the disclosure of which is included herein by reference in its entirety, discloses a wearable health monitoring system that continuously checks the wellbeing of a person that, typically, is considered healthy, covering a significant range of health hazards that may cause a significant life style change/limitation, and provides an alert as early as possible—all this, with no significant limitation to the normal life style of the person bearing the system.

Unlike conventional gel electrodes, which are directly applied to the living being's skin, using a conductive gel, textile electrodes are dry contact sensors adapted for use in measuring ECG signals and other vital signals such (EEG), electroencephalogram (EOG), electrooculogram and other medical measurements on the skin without any skin preparation, such as needed with wet electrodes, for example, shaving hairy skin

To improve performance over conventional wet ECG sensors and to be able to conduct continuous long term monitoring, a textile substrate is used to develop dry textile electrodes for sensing physiological parameters of a living being such as ECG signals. One such textile electrodes are disclosed in PCT application PCT/IL2013/050964, filed Nov. 23, 2013, titled “float loop textile electrodes and methods of knitting thereof”, the disclosures of which is included herein by reference for all purposes as if fully set forth herein.

There is however a need to transfer the sensed electrical signals from the textile electrodes to a processing unit for collecting and processing the sensed data.

Reference is made to FIG. 1 (prior art) depicting an open smart garment 20, having multiple textile electrodes 50 integrally knitted therein. Smart garment 20 is configured to receive a processing unit 70. FIG. 1 demonstrates the need to electrically connect each of the textile electrodes 50 to processing unit 70.

One solution is to integrally knit conductive traces form each of the textile electrodes 50 to a docking station configured to receive processing unit 70. This solution is disclosed in PCT application PCT/IL2013/050963, titled “vertical conductive textile traces and methods of knitting thereof”, filed Nov. 23, 2013, the disclosures of which is included herein by reference for all purposes as if fully set forth herein.

FIG. 2a (prior art) schematically illustrates an exemplary garment 20, having a tubular form, wherein textile electrodes 50 are knitted therein and are individually operatively connected to a processing unit 70. FIG. 2b (prior art) depicts a front view of an exemplary garment, wherein the textile electrodes 50 are designed to measure a 15-lead ECG signal, and are connected to a processing unit (not shown) by respective conductive traces 60.

The conductive traces 60 are knitted therein as part of the fabrication of the garment, wherein the conductivity, in particular between adjacent knitting courses in the vertical direction, can support the transfer of clinical level ECG signals from a textile electrode, along the fabric, to a selected area in the garment preconfigured to host the processing unit. Since the normal knitting direction of a tubular form is substantially horizontal, conductive traces 90 that are knitted therein in a horizontal direction maintain a stable conductivity.

The good conductivity should prevail when the fabric is stretched to different directions during wearing, which typically requires that the conductive physical means for transferring the sensed electrical signals from textile electrodes 50 to processing unit 70. This may entail that the conductive physical means is made of materials having high elasticity. This may entail that good conductive should prevail when the fabric is stretching, in particular between adjacent knitting courses in the vertical direction.

The good conductivity of the conductive physical means should prevail when using any type of basic fabric yarns (cotton, manmade yarns, synthetic yarns, metallic yarns, etc.).

The good conductivity should prevail after a preconfigured number of washes, including in a washing machine.

The good conductivity should prevail in any knitting design, location and shape in the fabric.

More so, signals detecting is the motion artifact occurring during movement of the person 10, wearing garment 20. The motion artifact problem may increase as a result of the large area of the textile electrodes 50 and/or the conductive traces 60, moving with respect to the skin of user 10. It should be noted that the larger the area of the textile electrodes 50 and/or the conductive traces 60 is, the higher the capacitance between the skin and textile electrode 50 and conductive traces 60 is.

There is therefore a need and it would be advantageous to provide conductive physical means for transferring the sensed electrical signals from textile electrodes to a target receiving unit that provides high conductivity and low sensitivity to motion artifacts.

### DEFINITIONS

The term “seamless monitoring”, as used herein with conjunction with wearable monitoring devices, refers to a device that when worn by an average person, wherein the device puts no significant limitation to the normal life style of that person and preferably not seen by anybody when used and not disturbingly felt by the user while wearing it. Furthermore, no activity is required from the monitored person in order for the system to provide a personal-alert when needed. It should be noted that people that pursue non-common life style, such as soldiers in combat zone or in combat training zone, or firefighters in training and action, or athletes in training or competition may utilize non-seamless monitoring devices. As the “seamless monitoring” characteristics refers also to the user's behavior, the wearable component is preferably an item that is normally worn (e.g., underwear) and not some additional item to be worn just for getting the alert. It should be noted that the term “seamless monitoring” differ from the notion of commonly known notion of a seamless clothing item that refers to tubular form clothing having no seams for forming the tubular form.

The terms “underwear” or “garment”, as used herein with conjunction with wearable clothing items, refers to wearable clothing items with seamless monitoring capabilities that preferably, can be tightly worn adjacently to the body of a monitored living being, typically adjacently to the skin, including undershirts, sport shirts, brassiere, underpants, special hospital shirt, socks and the like. Typically, the terms “underwear” or “garment” refer to a clothing item that is worn adjacently to the external surface of the user's body, under external clothing or as the only clothing, in such way that the fact that there are sensors embedded therein, is not seen by any other person in regular daily behavior. An underwear item may also include a clothing item that is not underwear per se, but still is in direct and preferably tight contact with the skin, such as a T-shirt, sleeveless or sleeved shirts, sport-bra, tights, dancing-wear, and pants. The sensors, in such a case, can be embedded in such a way that are still unseen by external people to comply with the “seamless monitoring” requirement.

The terms “course” and “line segment”, are used herein as related terms. The tubular form of the garment is knitted on a knitting machine, such as a Santoni knitting machine, where the tubular form is knitted in a spiral having substantially horizontal lines. A single spiral loop/circle us referred to herein as a course and a portion of a course is referred to as line segment.

The term “vertical conductive trace”, is used herein, refers to knitting a lead wire, made of conductive yarns, and capable of transferring electrical signals across knitted line segment.

The phrase “clinical level ECG”, as used herein with conjunction with ECG measurements, refers to the professionally acceptable number of leads, sensitivity and specificity needed for a definite conclusion by most cardiology physicians to suspect a risky cardiac problem (for example, arrhythmia, myocardial ischemia, heart failure) that require immediate further investigation or intervention. Currently, it is at least a 12-leads ECG and preferably 15-lead ECG, coupled with a motion/posture compensation element, and a real-time processor with adequate algorithms.

### BRIEF SUMMARY OF THE INVENTION

A principle intention of the present invention is to provide conductive physical means for transferring the sensed electrical signals from textile electrodes to a target receiving unit. Typically, the conductive physical means is composed of elastic conductive yarns, herein referred to as a “conductive stripe”. The conductive stripe is made of yarns selected form a group of yarns including manmade yarns, synthetic yarns and metallic yarns. The conductive stripe provides high conductivity, elasticity and low sensitivity to motion artifacts.

Another principle intention of the present invention is to connect textile electrodes to a signal receiving unit by a flexible and loose conductive stripe, such that the conductive stripe does not apply pulling forces or applies minimal pulling forces on the textile electrode securely connected thereto. Thereby, during motion, the textile electrode remains stably in position with respect to the skin of the user, while the signals, such as ECG signals, transfer to a receiving unit such as a docking station.

It should be noted that the signals can be any sensed electric signals (e.g. respiration) and it is not restricted to ECG signals. It should also be noted that any non-horizontal angle can be knitted using this invention by a continuous sequence of vertical lines.

It should be further noted that with respect to the embodiments provided by PCT application PCT/IL2013/050963, the embodiments of the present invention show significant reduction of motion artifact when the user is in motion, due to the fact that the new conductive elastic stripes are attached to the basic garment only in a few points such as to prevents the pulling the respective electrodes, which pulling may create unnecessary friction of the textile electrode with the skin. Furthermore, the present invention provides embodiment that substantially reduce the quantity and cost of materials and labor.

According to the teachings of the present invention there is provided a knitted smart garment. The garment includes a tubular form having a preconfigured elasticity, typically varied elasticity, and at least one conductive textile electrode for sensing an electrical vital signal, such as a clinical-level ECG signal. The garment further includes at least one elastic conductive stripe, having a first end and a second end.

The first end of the at least one conductive stripe is securely and conductively attached to a respective conductive textile electrode, and the second end of the at least one conductive stripe is operatively connected with a processor.

The elasticity of the at least one conductive stripe is configured to prevent a pulling force from being applied to the respective conductive textile electrode, when the garment is stretched.

The at least one conductive stripe is insulated by insulation means, wherein the insulation means are selected from the group including at least one insulating adhered stripe (110), sleeves (170), non-conductive coating and non-conductive textile material that is knitted, weaved, braided or covered on the respective at least one conductive stripe.

The insulation means are designed not reduce the conductivity of the respective the at least one conductive stripe. The insulation means are further designed not reduce the elasticity of the respective the at least one conductive stripe.

Typically, the at least one conductive stripe is at least partially loose inside the respective insulation means.

The at least one conductive stripe is made of yarns selected form a group of yarns including manmade yarns, synthetic yarns and metallic yarns, or a combination thereof.

The second end of the at least one conductive stripe may be securely attached to a connector, such as, with no limitations, a HDMI connector. Alternatively, the second end of the second end of the at least one conductive stripe is securely attached to a docking station.

The garment may include a zipper, wherein said zipper is situated between the at least one textile electrode and a docking station, wherein the at least one conductive stripe passes through the continuous section of the garment, without crossing the zipper, and wherein the second end of said respective at least one conductive stripe or knitted line-trace is securely attached to the docking station.

### BRIEF DESCRIPTION OF THE DRAWINGS

The present invention will become fully understood from the detailed description given herein below and the accompanying drawings, which are given by way of illustration and example only and thus not limitative of the present invention, and wherein:

FIG. 1 (prior art) depicts an open smart garment, having multiple textile electrodes integrally knitted therein, wherein the smart garment is configured to receive a processing unit.

FIG. 2a (prior art) is a schematic illustration of an exemplary garment, having a tubular form, wherein textile electrodes are knitted therein.

FIG. 2b (prior art) depicts a front view of an exemplary garment, wherein the textile electrodes are designed to measure a 15-lead ECG signal.

FIG. 3a depicts segments of a number of conductive stripes, according to embodiments of the present invention, wherein the conductive stripes are covered by an insulating tube, showing an open end of the conductive stripes.

FIG. 3b depicts segments of a number of conductive stripes, as in FIG. 3a, showing the other end of the conductive stripes, which, in the shown example, are connected to an HDMI connector.

FIG. 4 illustrates an example smart garment, having multiple textile electrodes integrally knitted therein, wherein the conductive stripes are configured to transfer the sensed electrical signals from the textile electrodes to a processing unit configured to collect the sensed data, according to some embodiments of the present invention.

FIG. 5 illustrates an example method of securely connecting a conductive stripe to a respective textile electrode, according to some embodiments of the present invention.

FIGS. 6a and 6b illustrate example smart garments, having multiple textile electrodes connected to conductive stripes, wherein insulating sleeves are used to insulate the conductive stripes from being electrically shortened by an adjacent conductive stripe and/or the user's skin, according to some embodiments of the present invention.

FIGS. 6c and 6d depict another example garment, according to the methods shown in FIGS. 6a and 6b. FIG. 6c, illustrating the internal side of garment the garment, having multiple textile electrodes connected to respective conductive stripes.

FIG. 7 illustrates an example smart garment, having multiple textile electrodes connected to conductive stripes, wherein a lining is used to insulate the conductive stripes from being electrically shortened by the user's skin, according to some embodiments of the present invention.

FIG. 8 is a schematic illustration of an exemplary garment having a tubular form and being an undershirt having a zipper in the front side, wherein textile electrodes are knitted therein.

FIG. 9 is a schematic illustration the exemplary garment shown in FIG. 8, wherein the zipper is unzipped and the garment in a spread, unfolded form.

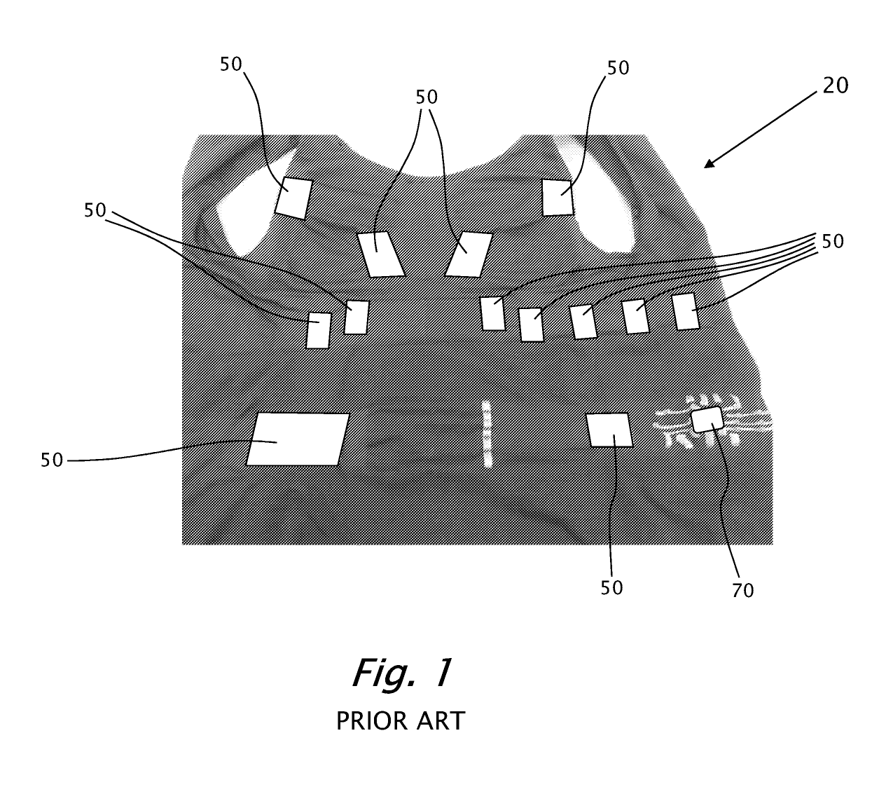

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art.

An embodiment is an example or implementation of the inventions. The various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments”, “another embodiment” or “other embodiments” means that a particular feature, structure, or characteristic described in connection with the embodiments is included in at least one embodiments, but not necessarily all embodiments, of the inventions. It is understood that the phraseology and terminology employed herein is not to be construed as limiting and are for descriptive purpose only.

Methods of the present invention may be implemented by performing or completing manually, automatically, or a combination thereof, selected steps or tasks. The term “method” refers to manners, means, techniques and procedures for accomplishing a given task including, but not limited to, those manners, means, techniques and procedures either known to, or readily developed from known manners, means, techniques and procedures by practitioners of the art to which the invention belongs. The descriptions, examples, methods and materials presented in the claims and the specification are not to be construed as limiting but rather as illustrative only.

It should be noted that orientation related descriptions such as “bottom”, “up”, “horizontal”, “vertical”, “lower”, “top” and the like, assumes that the is worn by a person being in a standing position.

Meanings of technical and scientific terms used herein are to be commonly understood as to which the invention belongs, unless otherwise defined. The present invention can be implemented in the testing or practice with methods and materials equivalent or similar to those described herein.

A principle intention of the present invention is to connect textile electrodes to a signal receiving unit by an elastic and loose conductive stripe, such that the conductive stripe does not apply pulling forces or applies minimal pulling forces on the textile electrode securely connected thereto. Thereby, during motion, the textile electrode remains stably in position with respect to the skin of the user, while the signals, such as ECG signals, transfer to a receiving unit such as a docking station.

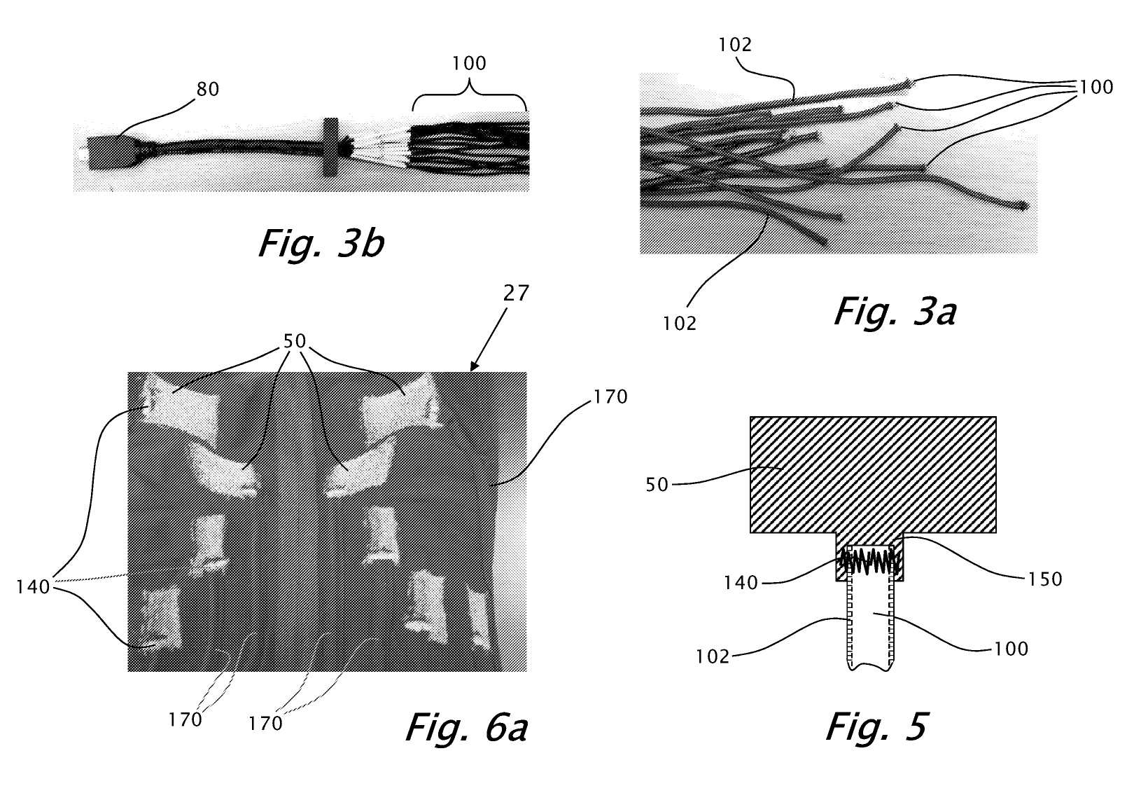

FIG. 3a depicts segments of a number of conductive stripes 100 that are covered by an insulating tube 102, showing an open end of conductive stripes 100. FIG. 3b depicts segments of a number of conductive stripes 100, showing the other end of conductive stripes 100, which in the shown example, with no limitation, are connected to an HDMI connector 80. Insulating tube 102 is elastic and does not limit the elasticity of conductive stripe 100.

Conductive stripes 100 can be made by knitting, weaving, braiding, or any other textile method which can combine both conductivity and elasticity. The good conductivity of conductive stripes 100 should prevail when using any type of basic fabric yarns to make the smart garment (such as manmade yarns, synthetic yarns, metallic yarns, etc.).

Conductive stripes 100 must be insulated to prevent electrical shorting among the stripes, while wearing and moving and to prevent conductive stripes 100 from being electrically shortened by the user's skin, by neighboring conductive stripes 100 or neighboring textile electrode 50.

The insulation can be done by knitting, weaving, braiding, and covering, using any non-conductive textile material, natural or synthetic yarns.

The insulation should not reduce the conductivity and the elasticity properties of conductive stripes 100.

Conductive stripes 100 are positioned in a preconfigured configuration along the shirt to facilitate the stripes to stretch while wearing.

In one embodiment of the present invention, the insulation of conductive stripes 100 is done after the braiding process, using Spandex yarn covered with Nylon yarn.

In one embodiment of the present invention, conductive stripes 100 are made of braided conductive yarns (for example, with no limitations, conductive yarns that are manufactured by XSTATIC) together with spandex yarns, in order to reach the right level of elasticity. However, conductive stripes 100 may be made using any other conductive materials such as stainless steel yarns, cooper yarns and any other combination of conductive yarns), provided that the of conductive stripes 100 is similar to the local elasticity of the smart garment.

The basic yarns to knit the smart garment and the type of Spandex yarn used should be in line with the machine gauge and type of fabric requested.

The quantity of conductive yarn ends (threads), elastic yarn ends, and the thickness (Den or Dtex) of the yarns in the braided stripe are determined by the level of conductivity and elasticity required for a particular smart garment.

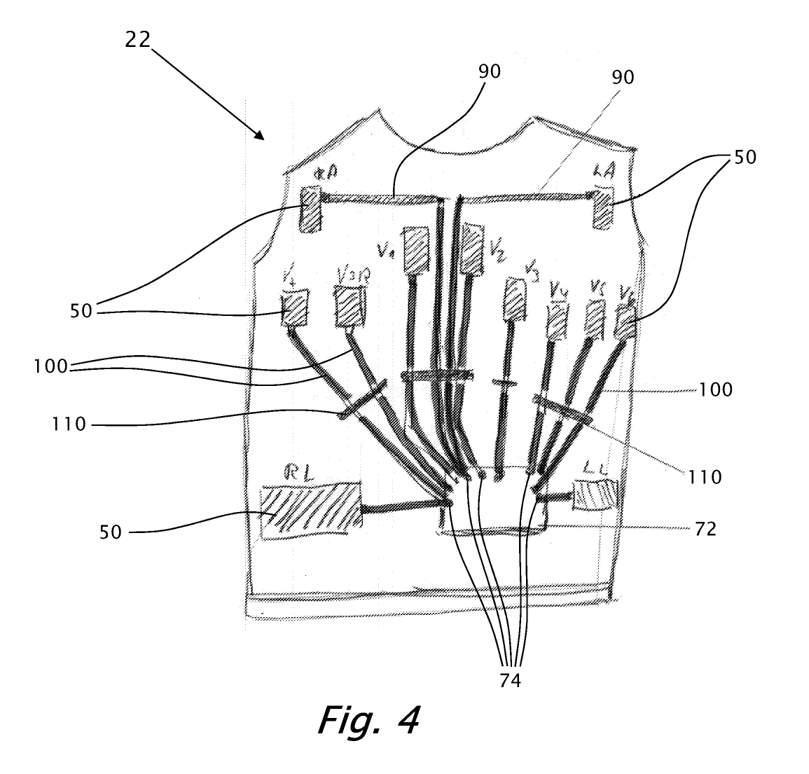

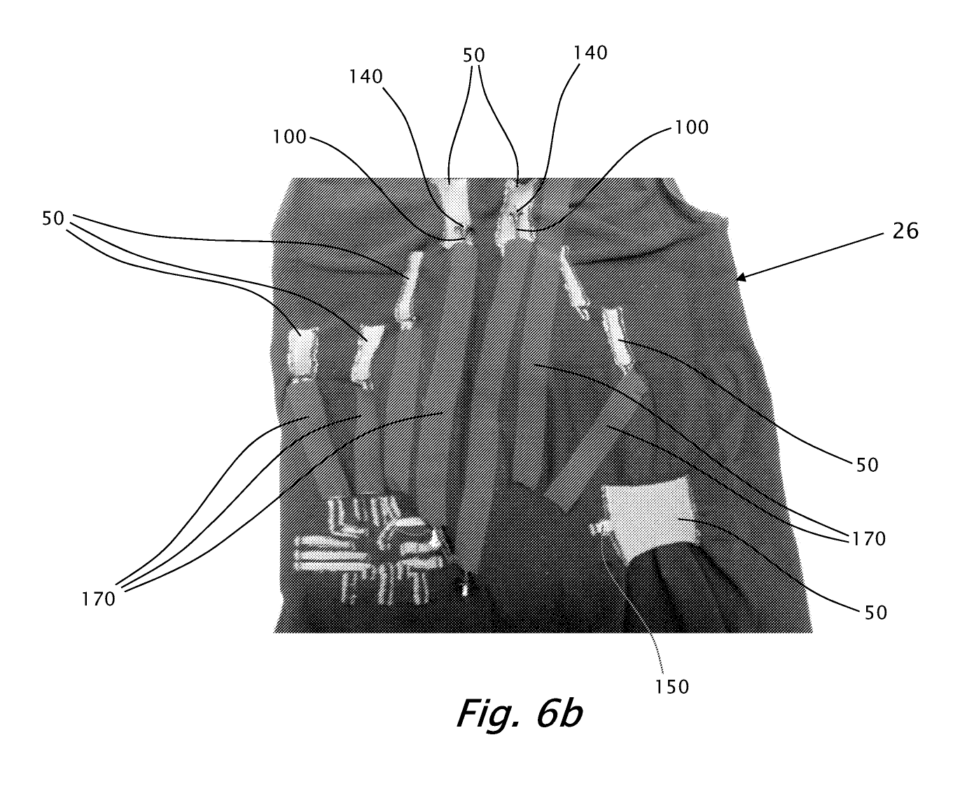

Reference is made to the drawings. FIG. 4 illustrates an example smart garment 22, having multiple textile electrodes 50 integrally knitted therein, wherein conductive stripes 100 are securely connected to respective textile electrodes 50, according to some embodiments of the present invention, facilitating the transfer of the sensed electrical signals from textile electrodes 50 to a target receiving unit such as a processing unit or a docking station 72. FIG. 5 illustrates an example method of securely connecting a conductive stripe 100 to a respective textile electrode 50, according to some embodiments of the present invention.

Smart garment 22, as shown by way of example only, with no limitations, as a knitted ECG shirt having 13 knitted electrodes (to all shown) at preconfigured locations on the shirt. Each of the knitted electrodes detects an ECG signal that is transferred to the receiving unit.

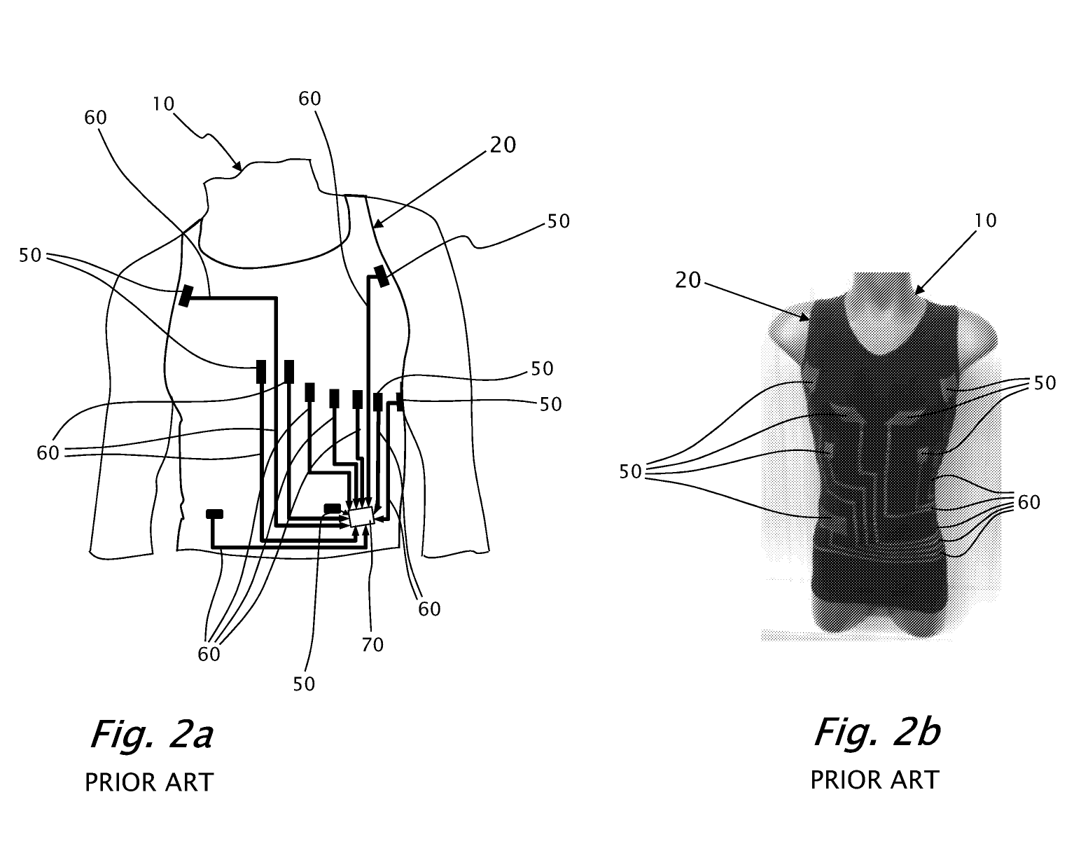

Each elastic conductive stripe 100 of smart garment 22 is attached to smart garment 22 at least three at points: securely attached to textile electrode 50, securely attached or passed through individual loops formed by a respective insulating adhered stripe 110, generally at middle area of smart garment 22, and securely connected to the receiving unit the a respective location, being, in the example shown in FIG. 2, a respective snap 74 of docking station 72.

Elastic conductive stripes 100 are attached to smart garment 22 leaving enough free length hanging loosely between points to allow the garment fabric to stretch during wear without pulling the respective textile electrode 50.

The mechanical attachment of elastic conductive stripe 100 to textile electrode 50 must ensure the smooth and efficient transfer of the clinical level ECG signal from the textile electrode 50 to the respective conductive stripe 100. For example, as shown in FIG. 5, conductive stripe 100 is sawn (140) to the respective textile electrode 50 at lingula 150. Conductive stripe 100 may also be attached to the respective textile electrode 50 by lamination (adhesion) or by heat press. The attachment means does not reduce the conductivity of either the textile electrode 50 or the respective conductive stripe 100.

It should be noted that conductive stripes 100 may be attached to the shirt at the inner or the outer sides of smart garment 22.

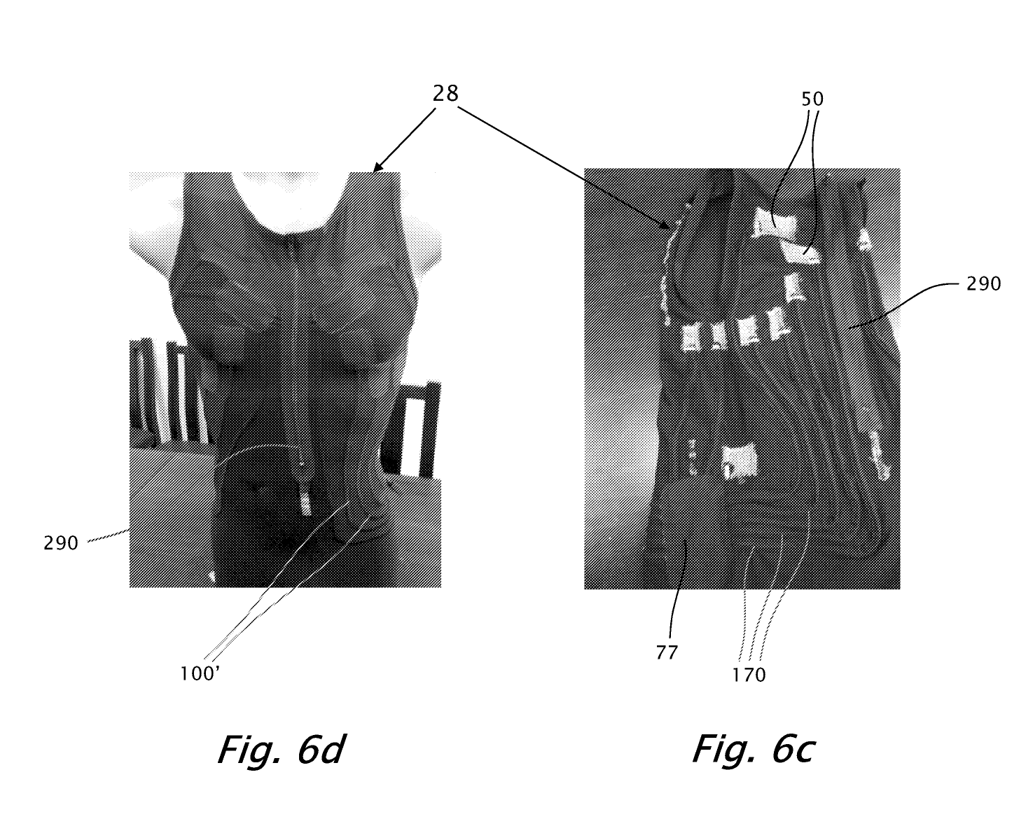

In some other embodiments of the present invention, each individual insulated conductive stripe 100 is inserted into a respective elastic sleeve which is securely attached to the fabric of the smart garment, for example by lamination. Reference is made to FIGS. 6a and 6b, depicting example methods of securely connecting a conductive stripe 100 to a respective textile electrode 50, according to other embodiments shown in FIG. 5. FIG. 6b, illustrates an example smart garments 26 and 27 (which garment 27 includes a zipper), having multiple textile electrodes 50 connected to conductive stripes 100, wherein insulating sleeves 170 are used to insulate conductive stripes 100 from being electrically shortened by an adjacent conductive stripe and/or the user's skin.

All conductive stripes 100 are inserted into respective sleeves 170, wherein one end of the elastic conductive stripe 100 is securely connected, for example by sewing, to a textile electrodes 50 and the other end of conductive stripe 100 is securely connected to a receiving unit, such as a docking station 72.

The usage of a laminated sleeve 170 for each of the conductive stripes 100, eliminates the usage of lining 160 to cover all conductive stripes 100, and keeps each conductive stripe 100 in a preconfigured path along the fabric of the smart garment (26 and 27).

FIGS. 6c and 6d depict another example garment 28, according to the methods shown in FIGS. 6a and 6b. FIG. 6c, illustrates the internal side (i.e., the skin side) of garment 28 (which garment 28 is a ladies garment that includes a zipper), having multiple textile electrodes 50 connected to respective conductive stripes 100, wherein insulating sleeves 170 are used to insulate conductive stripes 100 from being electrically shortened by an adjacent conductive stripe and/or the user's skin FIG. 6d illustrates the external side of garment 28 showing the protrusions 100′ formed by the sawn-in (on the internal side of garment 28) conductive stripes 100.

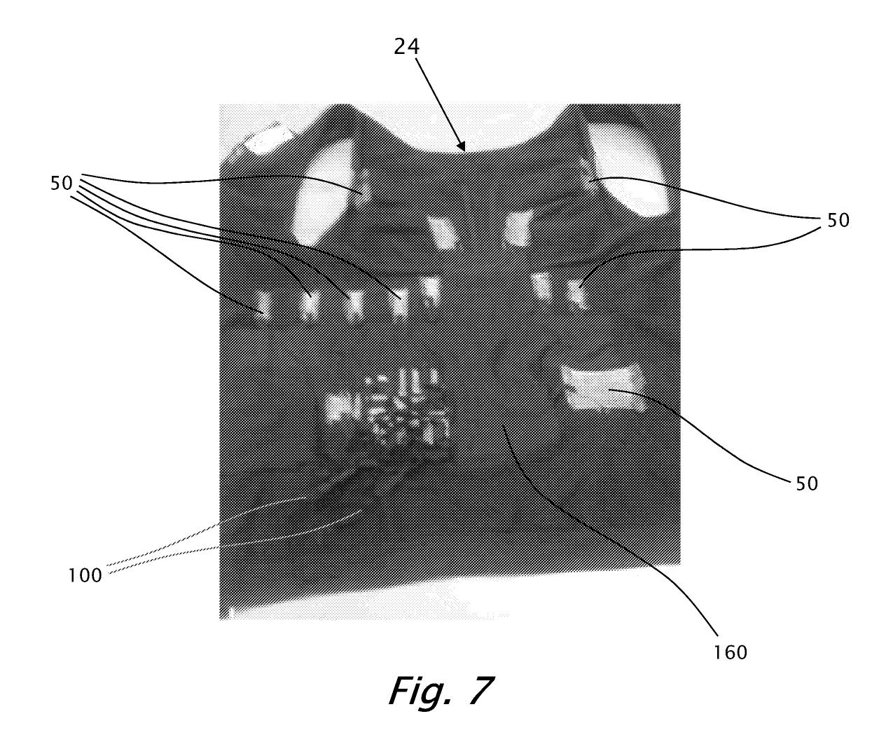

Reference is now also made to FIG. 7, showing an example smart garment 24, having multiple textile electrodes 50 connected to conductive stripes 100, wherein a lining 160 at the inner side of smart garment 24, wherein lining 160 is used to insulate conductive stripes 100 from being electrically shortened by the user's skin, according to some embodiments of the present invention Lining 160 facilitates each conductive stripe 100 to reach the right location 74 (see FIG. 4) at docking station 72.

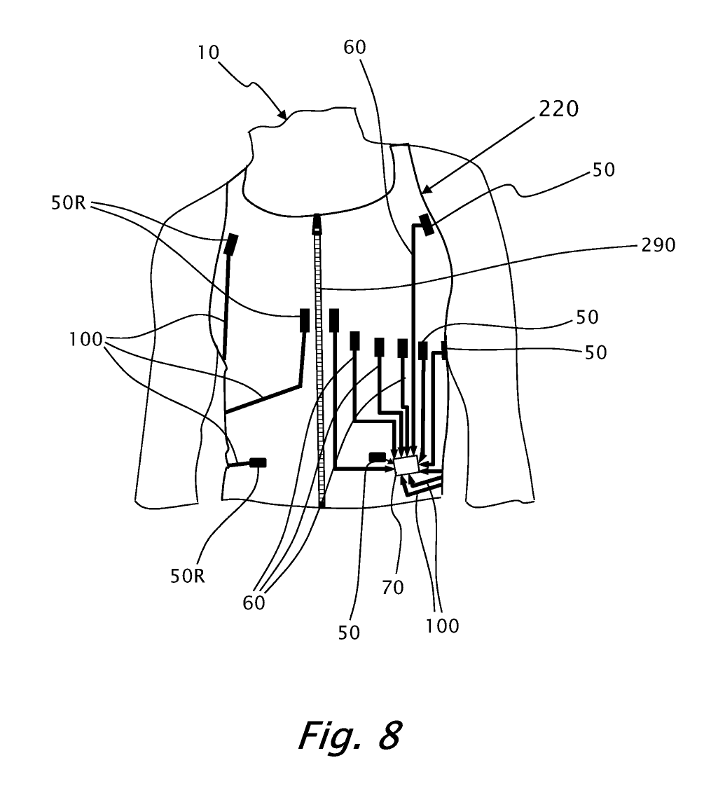

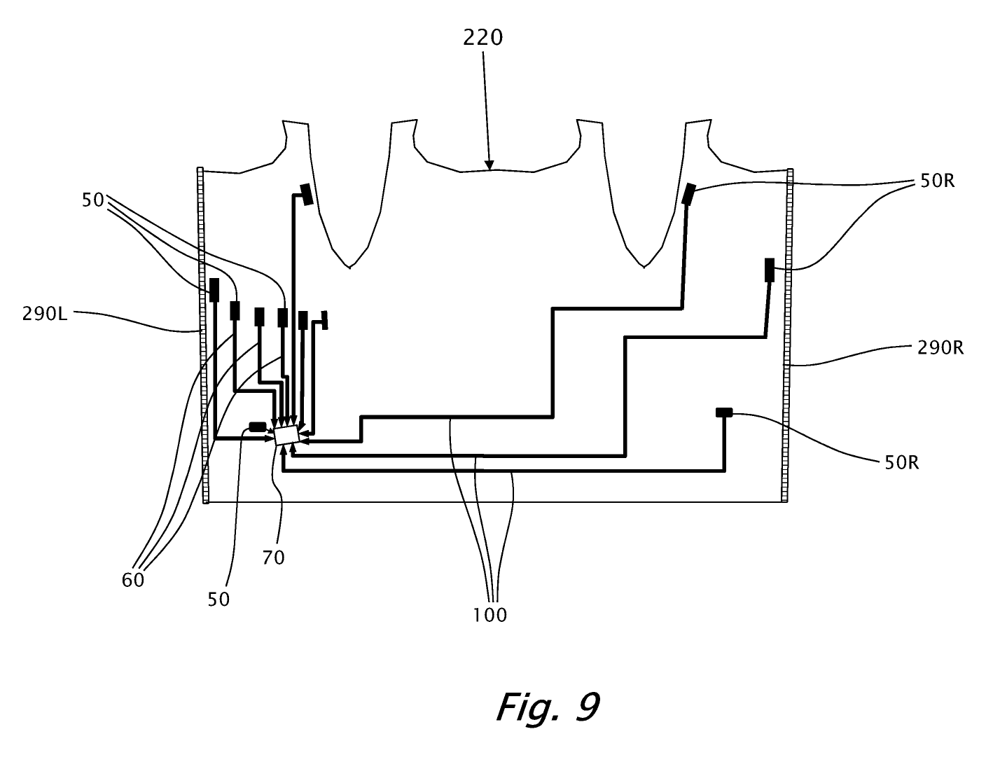

Reference in now made to FIG. 8, a schematic illustration of an exemplary garment 220 having a tubular form, the garment being an undershirt having a zipper 290 in the front side, wherein textile electrodes 50 are knitted therein and are individually operatively connected to processing unit 70. However, some electrodes, such as textile electrodes 50R, may require crossing zipper 290. To overcome the problem conductive stripes 100 or line-traces (not shown) are knitted into or attached to smart garment 220 in a path that is traced around, via the back side of the garment, such as to bypass zipper 290. FIG. 9 is a schematic illustration of an exemplary garment 220, as shown in FIG. 8, wherein zipper 290 is unzipped and the garment is in a spread, unfolded form.

The bypassing technique is also valid to any location of a generally vertical zipper, whereas conductive stripes 100 or knitted line-traces (not shown) are knitted into or attached to smart garment 220 in a path that is set to continuously pass through the continuous section of the garment between the 290L and 290R parts of zipper 290.

The invention being thus described in terms of embodiments and examples, it will be obvious that the same may be varied in many ways. Such variations are not to be regarded as a departure from the spirit and scope of the invention, and all such modifications as would be obvious to one skilled in the art are intended to be included within the scope of the claims.

1. A knitted smart garment, the garment comprising:
a) a tubular form having a preconfigured elasticity;
b) at least one conductive textile electrode for sensing an electrical vital signal; and
c) at least one elastic conductive stripe, having a first end and a second end,
wherein said first end of each said conductive stripe is securely and conductively attached to a respective said conductive textile electrode, and said second end of said at least one conductive stripe is operatively connected to a processor; and
wherein said elasticity of each said at least one conductive stripe is configured to prevent a pulling force from being applied to said respective conductive textile electrode, when said garment is stretched.

2. The garment of claim 1, wherein said electrical vital signal is a clinical-level ECG signal.

3. The garment of claim 1, wherein said at least one conductive stripe is insulated by insulation means.

4. The garment of claim 1, wherein said at least one conductive stripe movements are restricted by motion restricting means, wherein said motion restricting means are selected from a group of motion restricting means including sleeves (170) and sawn-in threads that are sawn over said at least one conductive stripe, and wherein said motion restricting means are securely attached to said garment.

5. The garment of claim 4, wherein said motion restricting means are securely attached to the external side of said garment.

6. The garment of claim 3, wherein said insulation means are selected from the group including at least one insulating adhered stripe (110), sleeves (170), non-conductive coating and non-conductive textile material that is knitted, weaved, braided or covered on the respective at least one conductive stripe.

7. The garment of claim 3, wherein said insulation means are designed not to reduce the conductivity of the respective said at least one conductive stripe.

8. The garment of claim 3, wherein said insulation means are designed not to reduce the elasticity of the respective said at least one conductive stripe.

9. The garment of claim 3, wherein said at least one conductive stripe is at least partially loose inside said insulation means.

10. The garment of claim 1, wherein said at least one conductive stripe is made of yarns selected form a group of yarns including manmade yarns, synthetic yarns and metallic yarns, or a combination thereof.

11. The garment of claim 1, wherein said second end of said at least one conductive stripe is securely attached to a connector.

12. The garment of claim 1, wherein said second end of said at least one conductive stripe is securely attached to a docking station.

13. The garment of claim 1 further comprising a zipper, wherein said zipper is situated between said at least one textile electrode and a docking station, wherein said at least one conductive stripe passes through the continuous section of the garment, without crossing said zipper, and wherein said second end of said respective at least one conductive stripe or knitted line-trace is securely attached to said docking station.
